(19) United States
(12) Patent Application Publication
(10) Pub. No.: US 2015/0088203 A1
(43) Pub. Date: Mar. 26, 2015

(54) METHOD AND DEVICE FOR BONE GROWTH STIMULATION

(71) Applicant: Sloan Robert, Indianapolis, IN (US)

(72) Inventor: Robert Sloan, Indianapolis, IN (US)

(21) Appl. No.: 14/036,905

(22) Filed: Sep. 25, 2013

(51) Int. Cl. A61B 17/70

(52) U.S. Cl. CPC A61B 17/7049
USPC 606/267; 606/279

(57) ABSTRACT

A device for attachment to a spinal implant construct is disclosed. The device generally includes at least one pedicle screw with a housing adapted for subcutaneous implantation. The housing comprises an electrical battery and an electrically conductive attachment to conduct electrical current from the battery to the screw. Once the screw is inserted into the patient's pedicle, the housing and attachment are inserted in the patient either by minimally invasive tubing or through a minimally invasive incision. The attachment may be attached to the implant construct after the pedicle screw has been inserted into the patient's pedicle.

### BACKGROUND

The present invention relates to an attachment to a spinal implant construct, and more specifically to electro-stimulation.

Electro-stimulation is known to facilitate bone growth. It has been used in various devices, including U.S. Pat. No. 7,935,116 B2 (Michelson, 2011) with an inter-vertebral cage and U.S. Patent Application 2009/0054951 A1 (Leuthardt et al., 2009) with a set-screw attaching a wire to a bone screw. However, most, if not all, commercially viable electro-bone growth stimulation systems include ex-corporeal power sources, using electro-magnetic fields through the skin or percutaneous leads. These can be cumbersome and lead to non-compliance by patients.

The present invention enhances bone growth without need for much, if any, patient compliance.

Thus, there is a need for improvement in this field.

### SUMMARY

The claims, and only the claims, define the invention. The invention may be an attachment to a spinal implant construct including at least one pedicle screw. The attachment has a housing adapted for subcutaneous implantation, an electrical battery within the housing, and an electrically conductive attachment for conducting electrical power from the battery to the spinal implant construct.

The present invention may also include a surgical method of attaching the spinal implant construct to a patient and subsequently attaching the housing to the spinal implant construct.

Further forms, objects, features, aspects, benefits, advantages, and embodiments of the present invention will become apparent from a detailed description and drawings provided herewith.

### BRIEF DESCRIPTION OF THE DRAWINGS

FIG. 1 is an embodiment of the spinal implant system.

FIG. 2 is a side view of an embodiment of the housing and conductive attachment device attached to the connector.

FIG. 3A is a view of an embodiment of the housing and conductive attachment device.

FIG. 3B is a view of another embodiment of the housing and conductive attachment device that snaps onto the object to which it is to be attached.

FIG. 4 is a top view of an embodiment of the housing and the actuator opening.

FIG. 5 is a bottom view of an embodiment of the housing and conductive attachment device.

FIG. 6 is an embodiment of the spinal implant system in which the electrically conductive attachment is pre-attached to the connector.

FIG. 7A is a cross-sectional view of the housing and conductive attachment in which the threaded member has not been advanced within the housing.

FIG. 7B is a cross-sectional view of the housing and conductive attachment in which the threaded member has been partially advanced within the housing.

FIG. 7C is a cross-sectional view of the housing and conductive attachment in which the threaded member has been fully advanced within the housing.

FIG. 8A is a flowchart for an embodiment of a method of installing the implant system when there are multiple screws and the housing and conductive attachment device are not pre-attached.

FIG. 8B is a flowchart for an embodiment of a method of installing the implant system when there are multiple screws and the housing and conductive attachment device are pre-attached.

FIG. 9 is an embodiment of the connector that includes electric current paths that provide electric current to each screw.

### DESCRIPTION OF THE SELECTED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail, although it will be apparent to those skilled in the relevant art that some features that are not relevant to the present invention may not be shown for the sake of clarity.

As used here (claims, specification, and other definitions) the following terms have the following meaning:

Articles and phases such as, “the”, “a”, “an”, “at least one”, and “a first”, “comprising”, “having” and “including” here are not limited to mean only one, but rather are inclusive and open ended to also include, optionally, two or more of such elements and/or other elements. In terms of the meaning of words or terms or phrases herein, literal differences therein are not superfluous and have different meaning, and are not to be synonymous with words or terms or phrases in the same or other claims.

The term “and/or” is inclusive here, meaning “and” as well as “or”. For example, “P and/or Q” encompasses, P, Q, and P with Q; and, such “P and/or Q” may include other elements as well.

The term “spinal implant system” as used herein means a structural assembly of multiple parts that is inserted into the body and attached to the spine.

The term “implant system” as used herein means a structural assembly of multiple parts that is inserted into the body and attached to one or more bones.

The term “rod” as used herein means a straight length of material used to span two points. This may be made of metal, plastic, or any other kind of material. Typically, it is primarily made of titanium or stainless steel. The cross-section is typically cylindrical, but may take other shapes such as square, triangular, or otherwise.

The term “pedicle” as used herein means the portion of the spine which projects dorsally from the body of a vertebra.

The term “screw” as used herein means a fastener with cancellous threads for purchase in bone tissue.

The term “housing” as used herein means a structure having an exterior portion or wall to cover and protect its interior.

The term “adapted” as used herein means shaped and sized to serve a function or purpose within its environment.

The term “subcutaneous implantation” as used herein means implantation under the skin.

The term “electrical battery” as used herein means any device that converts chemical energy or any other form of energy into electrical energy.

The term “electrically conductive attachment” as used herein means an object that connects to another object and allows for the substantial flow of electric current. The connection may be permanent or temporary. The attachment may be made of any material that conducts electricity, but normally is metal.

The term “electrical current” as used herein means the flow of electrical charge or electrons.

The term “footprint” as used herein means the surface area that is occupied by the area at the largest area cross-section or the widest cross-sectional diameter of an object.

The term “minimally invasive tube” as used herein means a hollow instrument that can be inserted into a minimally invasive incision that allows material to be passed through the incision without contacting any part of the body or body fluids. The minimally invasive tube may be circular, square, or any other shape that may fit through the incision. It may be constructed of metal, plastic or any other material.

The term “minimally invasively inserted” as used herein means inserted through a patient's tissue using minimally invasive surgical techniques.

The term “minimally invasive incision” as used herein mean one or more cuts made for the purpose of performing a minimally invasive procedure. The cutting is smaller than would be made for open surgery done for the same purpose.

The term “electrically insulated” as used herein means that objects are separated in some manner so that electric current may not be conducted between them. This includes separation by a material that nonconductive material such as air, plastic, fiberglass, or otherwise.

The term “tissue” as used herein means non-bone tissue found in the human body. This includes skin, connective tissue, muscle tissue, nervous tissue, and epithelial tissue.

The term “attachable” as used herein means adapted to being secured to another object. The objects may be secured temporarily or permanently.

The term “connector” as used herein means any object that joins, links, or fastens together objects. A connector may take many forms such as a rod, a fitting, a screw, a nail, or otherwise. A connector may be made of any solid material that can join, link, or fasten objects together, for example, steel, plastic, or aluminum.

The term “non-toxic” as used herein means not containing any of the following: lead, mercury, cadmium, hexavalent chromium (chromium xxx or Cr6+), or polybrominated biphenyls (PBB). Above and beyond the materials restricted by RoHS, non-toxic is also used to mean free of heavy metals or any other toxic materials.

The term “anode” as used herein means an electric terminal at which electrons enter.

The term “cathode” as used herein means an electric terminal at which electrons flow out from.

The term “electric current paths” as used herein means any length of material that allows electric current to flow.

The term “threaded member” as used herein means any object with exterior threading so that it may be advanced in an interiorly threaded opening upon rotation. This may be a screw or any other object that has exterior threads.

The term “cams” as used herein means to cause movement with the use of a cam. A cam is a rotating or sliding linkage that upon rotation or sliding movement mechanically urges via contact (direct or indirect) another object to move.

The term “finger” as used herein means an extension of or from a part used for attaching to or in or contacting another object.

The term “inserter tool” as used herein means any tool used to place any part of the implant system within the body. This includes, but is not limited to, tools like tweezers or pliers or any other device that may insert a structure into the body. The inserter tool may be inserted into the internal portion of the body when in use or may be used solely exterior to the body.

The term “elongated shank” as used herein means the part of an inserter device or tool that is that is connected to the object being inserted on one end attached to the part of the tool held by the user on the other end.

The term “actuator” as used herein means a device used to cause attachment of the electrically conductive attachment to the implant system. This can be a button located on the attachment, a switch on that causes connection to the implant system, a snap that connects to the implant system when pressure is applied, a threaded screw that causes fingers to close around a rod, or any other object that initiates attachment.

The term “vertebral body” as used herein means the solid, central portion of a vertebra.

The term “means” and/or “means for” here, when used in a claim, invokes 35 U.S.C. §112 ¶6 means-plus-function for the recited function(s) and the corresponding structure(s) (including alternatives in the definitions or elsewhere in this disclosure) and equivalents thereto.

The language used in the claims and the written description and in the above definitions is to only have its plain and ordinary meaning, except for terms explicitly defined above. Such plain and ordinary meaning is defined here as inclusive of all consistent dictionary definitions from the most recently published (on the filing date of this document) general purpose Webster's dictionaries and Random House dictionaries.

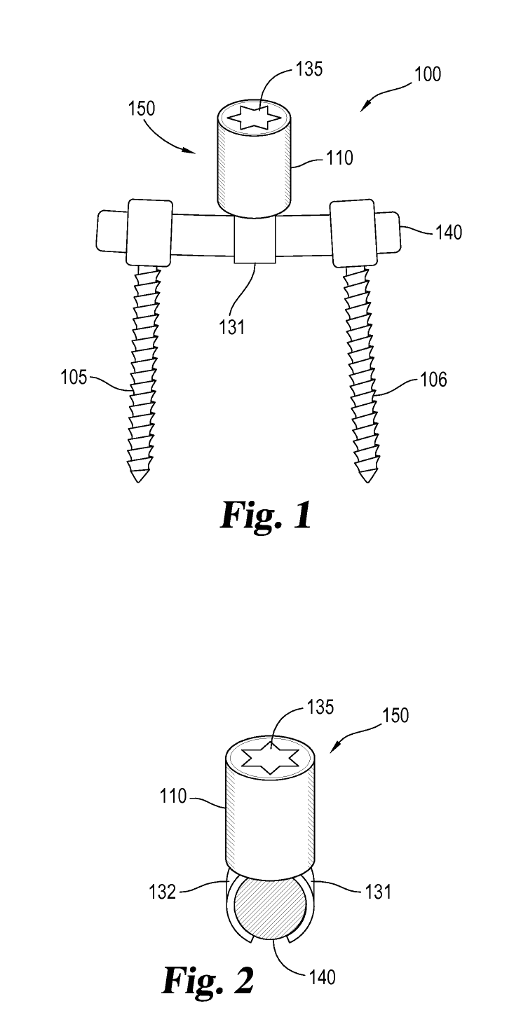

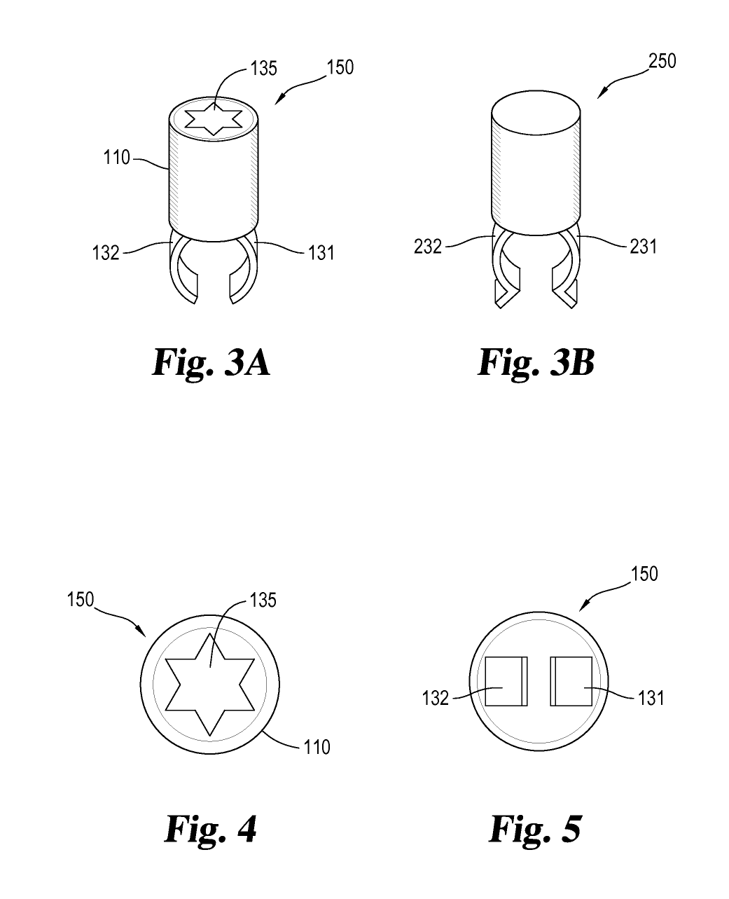

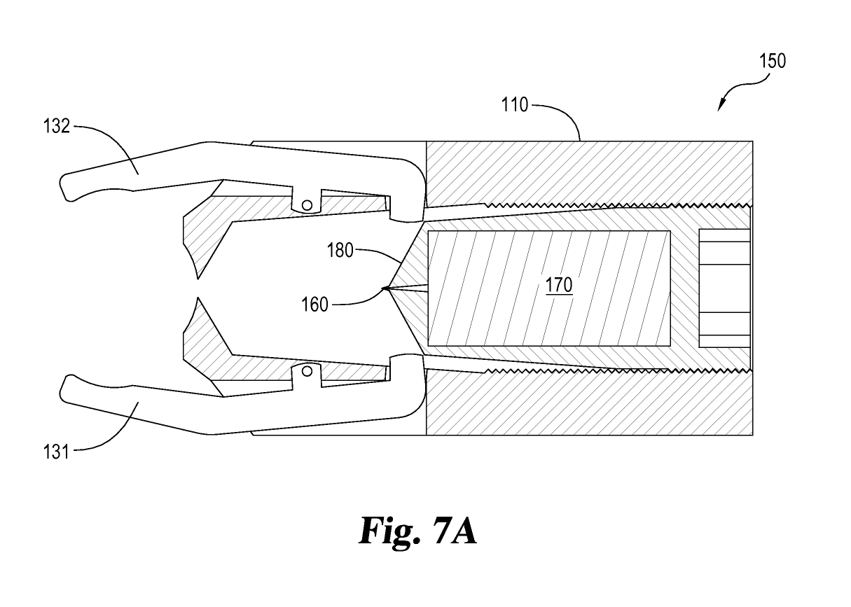

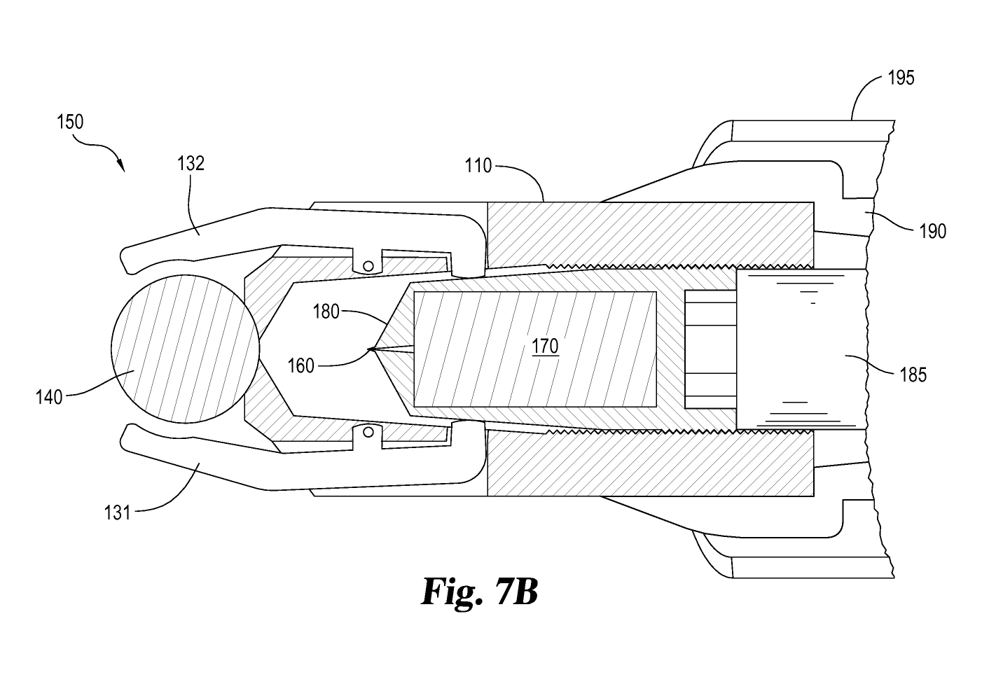

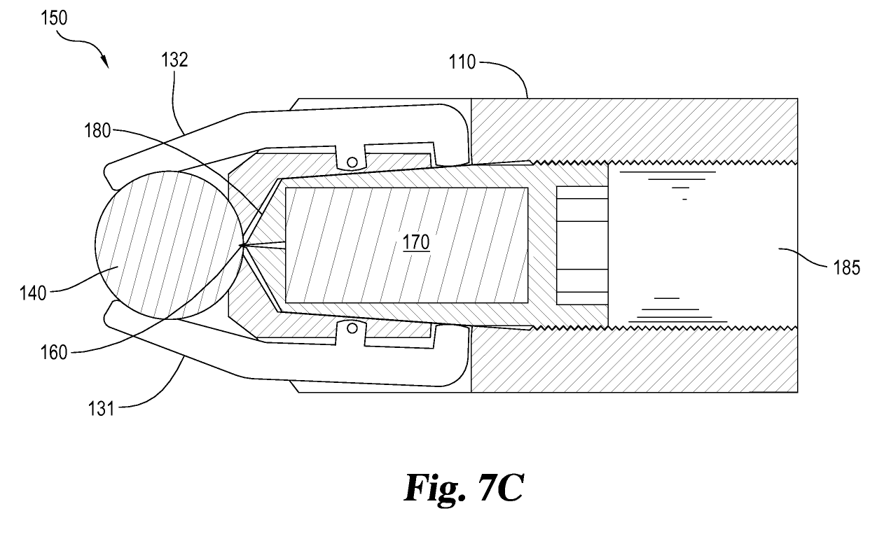

Referring to the drawing figures, these are only examples of the invention, and the invention is not limited to what is shown in the drawings. For example, FIG. 1 shows a housing and attachment device 150 (as shown in FIG. 3A) for attachment to a spinal implant system 100 which includes a first pedicle screw 105 and a second pedicle screw 106. In some embodiments connector 140 connects the first and second pedicle screws 105, 106 together. The spinal implant system 100 also contains a housing 110 adapted for subcutaneous implantation. An electrical battery 170 (as seen in FIG. 7A) is contained within the housing. The device also includes an electrically conductive attachment 160 that attaches to the connector 140 and conducts electrical current from the battery 170 to the connector 140 and pedicle screws 105, 106.

The housing and attachment device is sized and adapted to be minimally invasively inserted in a patient. Once the screws 105,106 have been inserted into the patient's pedicles, the insertion of the housing 110 and attachment device may be accomplished using various methods. Examples include using minimally invasive tubing or by making a minimally invasive incision through soft tissue. Insertion may include both minimally invasive tubing and making a minimally invasive incision. Because the implant system 100 is attached to the spine through the use of a minimally invasive procedure the components of the implant system 100 has a limited size. For example, in one embodiment, the cross-sectional footprint of the housing and attachment device 150 is limited to a maximum diameter of 30 millimeters.

An optional feature is where the spinal implant system includes at least a second pedicle screw and a connector adapted for connecting the first and second pedicle screws together (see e.g. FIG. 1), wherein the electrically conductive attachment is adapted to provide the electrical current along at least a portion of the connector to at least one of the pedicle screws.

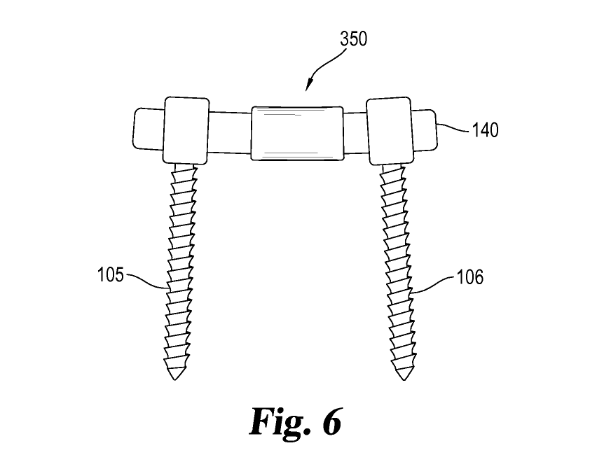

In another embodiment, the connector is a rod and the electrically conductive attachment is separate from but attachable to the rod (see e.g. FIG. 2). However, the electrically conductive attachment could optionally be pre-attached to the rod (as shown in FIG. 6).

Another optional feature is that the battery may be a non-toxic battery.

Another embodiment has at least one pedicle screw that acts as an anode pedicle screw and at least one more pedicle screw that comprises a cathode pedicle screw. Electrical current from the battery flows across tissue between the cathode pedicle screw and the anode pedicle screw.

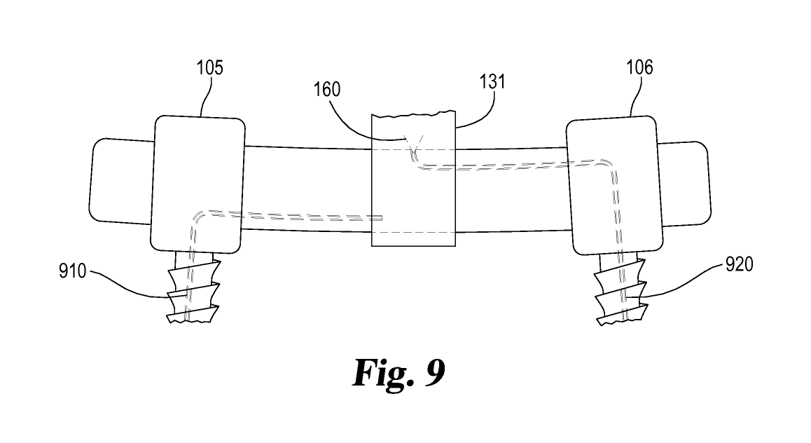

An optional feature (shown in FIG. 9) is the presence of at least two electric current paths 910, 920 which are electrically insulated from each other. The first path runs to the first pedicle screw and the second path runs to the second pedicle screw. These electric current paths may connect to the battery directly at the conductive attachment 160 or electric current may be run through the fingers 131, 132 to the electric current path.

An optional feature is an electrical controller which switches the polarity of the electrical current flow back and forth between the anode and the cathode pedicle screws.

Another optional feature is a threaded member 180 (see e.g. FIG. 7B or FIG. 7C) within the housing which axially advances when it is rotated. Upon advancement the threaded member cams at least one finger 131, 132 (shown from a bottom view in FIG. 5) of the attachment to close around the implant system.

The device may also include an inserter tool 190 which is attachable and detachable from the housing 110. The inserter tool comprises an elongated shank adapted to insert through either a minimally invasive tube 195, a small incision through muscle, or both; and, an actuator 185 that causes the conductive attachment to attach to implant system.

Optionally, the housing and attachment device has an actuator opening 135 on its top that engages with the actuator 185. This actuator opening may be a variety of shapes. For example, it could be a six sided star (see FIG. 4), a square, a Phillips head shape, or a slot.

Another optional feature is a housing and attachment device 250 (see FIG. 3B) that snaps onto the connector rather than being attached with an actuator. The fingers 231, 232 of this embodiment have flared ends that allow the device to snap onto the connector instead of needing to use an actuator to attach to the connector.

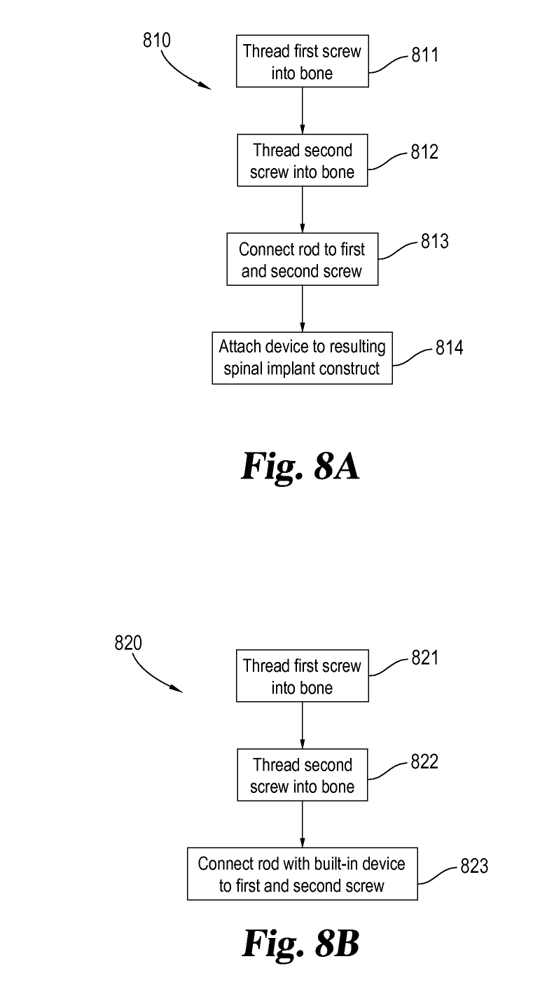

A surgical method for installing the implant system 810 is given (see e.g. FIG. 8A). The first step is to surgically thread a first screw through a first bone 810. Then a second screw is threaded through a second bone 811. Next, connect a rod to the first screw and the second screw to form an implant construct 812. Finally, attach a device posteriorily to the implant construct 813. The device comprises a housing adapted for subcutaneous implantation, an electrical battery within the housing, and an electrically conductive attachment for conducting electrical power from the battery to the implant construct.

An optional method 820 (see e.g. FIG. 8B) exists where the device is pre-attached to the connector. In this case, the first step is to surgically thread a first screw through a first bone 820. Then, a second screw is threaded through a second bone 821. Finally, connect the rod with the built-in device into the first screw and the second screw to form an implant construct 822.

Optionally, the method described above could pertain to a spinal implant construct. In this embodiment, the screws are pedicle screws that are threaded into pedicles and into the vertebral body.

While the invention has been illustrated and described in detail in the drawings and foregoing description, the same is to be considered as illustrative and not restrictive in character, it being understood that only the preferred embodiment has been shown and described and that all changes, equivalents, and modifications that come within the spirit of the inventions defined by following claims are desired to be protected. All publications, patents, and patent applications cited in this specification are herein incorporated by reference as if each individual publication, patent, or patent application were specifically and individually indicated to be incorporated by reference and set forth in its entirety herein.

What is claimed is:

1. A device for attachment to a spinal implant system including at least a first pedicle screw, the device comprising:
a housing adapted for subcutaneous implantation;
an electrical battery within said housing;
an electrically conductive attachment for conducting electrical current from said battery to the pedicle screw;
wherein, after insertion of the pedicle screw into a patient's pedicle, said housing and said attachment collectively are sized and adapted to be minimally invasively inserted in a patient via: minimally invasive tubing, minimally invasive incision through soft tissue, or both;
wherein said attachment is attachable to the implant system after the pedicle screw is implanted in a patient's pedicle to provide said electrical current to the pedicle screw.

2. The device of claim 1 wherein the spinal implant system includes at least a second pedicle screw and a connector adapted for connecting the first and second pedicle screws together, wherein said electrically conductive attachment is adapted to provide said electrical current along at least a portion of said connector to at least one of the pedicle screws.

3. The device of claim 1 wherein said battery is a non-toxic battery.

4. The device of claim 2 wherein at least one pedicle screw comprises an anode pedicle screw, and wherein at least another pedicle screw comprises a cathode pedicle screw, wherein electrical current from said battery flows across tissue between said cathode pedicle screw and said anode pedicle screw.

5. The device of claim 4 and further comprising an electrical controller which switches polarity of electrical current flow back and forth between said anode and cathode pedicle screws.

6. The device of claim 5 wherein said battery is a non-toxic battery.

7. The device of claim 1 wherein said housing and said attachment collectively have a cross-sectional footprint with a maximum cross-sectional diameter of 30 millimeters and is adapted to be inserted toward said spinal implant construct minimally invasively.

8. The device of claim 2 wherein said connector is a rod, and said electrically conductive attachment is separate from but attachable around and to the rod.

9. The device of claim 2 wherein said connector is a rod, and said electrically conductive attachment is pre-attached to said rod.

10. The device of claim 2 wherein said connector has at least two electric current paths which are electrically insulated from each other, wherein a first said path runs to said first pedicle screw and wherein said second said path runs to said second pedicle screw.

11. The device of claim 1 wherein said housing includes a threaded member which upon rotation axially advances and cams at least one finger of said attachment to close around said implant system.

12. The device of claim 1 and further comprising an inserter tool which is attachable and detachable from said housing, said inserter tool including: (a) an elongated shank adapted to insert through either a minimally invasive tube, a small incision through muscle, or both; and, (b) an actuator to cause said conductive attachment to attach to implant system.

13. A device for attachment to an implant system including at least a first screw, the device comprising:
a housing adapted for subcutaneous implantation;
an electrical battery within said housing;
an electrically conductive attachment for conducting electrical current from said battery to the screw;
wherein said housing includes a threaded member which upon rotation axially advances and cams at least one finger of said attachment to close around the implant system.

14. The device of claim 13 wherein the implant system includes at least a second screw and a connector adapted for connecting the first and second screws together, wherein said electrically conductive attachment is adapted to provide said electrical current along at least a portion of said connector to at least one of the screws.

15. The device of claim 14 wherein at least one screw comprises an anode screw, and wherein at least another screw comprises a cathode screw, wherein electrical current from said battery flows across tissue between said cathode screw and said anode screw.

16. The device of claim 14 and further comprising an electrical controller which switches polarity of electrical current flow back and forth between said anode and cathode screws.

17. A surgical method, comprising the acts of:
(a) Surgically:
Threading a first pedicle screw through a first pedicle and into a vertebral body;
Threading a second pedicle screw through a second pedicle and into a vertebral body;
Connecting a rod to said first pedicle screw and said second pedicle screw to form a spinal construct; and,
(b) Thereafter, surgically:
Attaching a device posteriorily to said spinal implant construct, the device comprising:
a housing adapted for subcutaneous implantation;
an electrical battery within said housing;
an electrically conductive attachment for conducting electrical power from said battery to the spinal implant construct.

18. The method of claim 15 wherein said housing and said attachment collectively have a cross-sectional footprint with a maximum cross-sectional diameter of 30 millimeters and is adapted to be inserted toward said spinal implant construct minimally invasively through either a minimally invasive tube, a minimally invasive incision through tissue, or both; and,
wherein said attachment is mechanically and electrically connectable to said spinal implant construct.

19. The method of claim 15 wherein said housing includes a threaded member which upon rotation axially advances and cams at least one finger of said attachment to close around the rod.

20. The method of claim 15 wherein at least one pedicle screw comprises an anode pedicle screw, and wherein at least another pedicle screw comprises a cathode pedicle screw, wherein electrical current from said battery flows across tissue between said cathode pedicle screw and said anode pedicle screw.

21. The method of claim 18 and further comprising an electrical controller which switches polarity of electrical current flow back and forth between said anode and cathode pedicle screws.
